(19) United States
(12) Patent Application Publication
(10) Pub. No.: US 2026/0144975 A1
(43) Pub. Date: May 28, 2026

(54) CONTINUOUS SEALING TUOHY-BORST ADAPTER

(71) Applicant: CygnusMed, Inc., Hudson, OH (US)

(72) Inventor: Firas Al-Ali, Hudson, OH (US)

(73) Assignee: CygnusMed, Inc., Hudson, OH (US)

(21) Appl. No.: 19/453,510

(22) Filed: Jan. 20, 2026

Related U.S. Application Data
(63) Continuation-in-part of application No. 19/070,749, filed on Mar. 5, 2025.
Application No. 19/070,749 is a continuation-in-part of application No. 17/575,780, filed on Jan. 14, 2022, now Pat. No. 12,447,324.
Application No. 17/575,780 is a continuation-in-part of application No. 16/784,735, filed on Feb. 7, 2020, now Pat. No. 11,376,408.
(63) Continuation of application No. 17/575,783, filed on Jan. 14, 2022.
(60) Provisional application No. 63/187,074, filed on May 11, 2021.

(51) Int. Cl. A61M 39/06; A61M 39/22; A61M 39/24

(52) U.S. Cl. CPC A61M 39/0613; A61M 39/24; A61M 2039/062; A61M 2039/0666; A61M 2039/0673; A61M 2039/229; A61M 2039/2406

(57) ABSTRACT

A dynamic radial seal for Touhy-Borst adapters is provided. The seal has a breaking pressure above typical pressure regimes encountered in vivo in the blood of human subjects. A Touhy-Borst adapter is also provided which incorporates the dynamic radial seal.

This application is a continuation-in-part of pending U.S. application Ser. No. 19/070,749 filed Mar. 5, 2025, which is a continuation-in-part of granted U.S. application Ser. No. 17/575,780 filed Jan. 14, 2022 now U.S. Pat. No. 12,447,324, which is a continuation-in-part of granted application Ser. No. 16/784,735 filed Feb. 7, 2020, now U.S. Pat. No. 11,376,408. Granted U.S. application Ser. No. 17/575,780 also claims the benefit of expired U.S. Provisional Application No. 63/187,074 filed May 11, 2021. Granted application Ser. No. 16/784,735 is a continuation-in-part of granted application Ser. No. 15/782,664 filed on Oct. 12, 2017 now U.S. Pat. No. 10,625,067, which claims the benefit of U.S. Provisional Application No. 62/407,258 filed Oct. 12, 2016. Granted application Ser. No. 15/782,664 is a continuation-in-part of granted U.S. application Ser. No. 15/907,429 filed Feb. 28, 2016 now U.S. Pat. No. 10,939,831. The present application is also a continuation-in-part of pending U.S. application Ser. No. 17/575,783 filed Jan. 14, 2022. All of the foregoing applications are hereby incorporated by reference in their entireties.

### I. BACKGROUND OF THE INVENTION

### A. Field of Invention

The present invention generally relates to the field of Tuohy-Borst adapters, especially the bleed-back control valves used therein for medical catheterization procedures.

### B. Description of the Related Art

Bleed-back control valves are well-known and have long been in use in surgical intervention and diagnostic procedures involving catheters. They are alternatively known as backflow control valves and hemostasis valves. One common bleed-back control valve is traditionally used in the Tuohy-Borst adapter. Tuohy-Borst adapters are used in catheterization procedures where a catheter is fed into the adapter through a distal catheter access port, it travels through the lumen of the adapter, and exits through another port at the proximal end, thus entering the patient. The terms distal and proximal are used herein according to the medical convention relative to the heart, where distal means further from the heart, and proximal means nearer the heart.

Tuohy-Borst adapters include a threaded fitting containing a compressible cylindrical gasket. As the gasket is axially compressed by the fitting, it collapses around the catheter, locking it in place and preventing blood or other fluids from backflowing through the catheter access port. The typical mode of using a Tuohy-Borst adapter is to feed a catheter through the adapter to position it within a patient. Once positioned, the catheter is locked in place.

The Tuohy-Borst adapter is a very common tool in the medical profession even to the extent of being a standard; however, this tool has certain long-standing shortcomings. For instance, bleed-back can only be stopped when the catheter is locked in place. Therefore, as the physician is positioning the catheter within a patient, blood will backflow to some extent. This creates a blood spill, which is undesirable because it increases the risk of exposure to blood-borne pathogens, and because blood loss can have negative consequences for the patient. Generally, the physician will loosen the catheter just enough to allow the catheter to slide. This tends to limit bleed-back, but it does not eliminate it. The bleed-back problem has significant health consequences for both the patient and the medical professionals treating the patient. Nonetheless, the problem has persisted unresolved since the Tuohy-Borst was first introduced in the mid-twentieth century.

What is needed is a dynamic radial seal for a bleed-back control valve that slideably engages a catheter while simultaneously blocking bleed-back. Some embodiments of the present invention may provide one or more benefits or advantages over the prior art.

### II. SUMMARY OF THE INVENTION

Embodiments of the invention include a dynamic radial Touhy-Borst seal having a base flange or other mounting means for cooperating with a Touhy-Borst adapter. The seal further includes a plurality of pliable occlusive elements radiating inward from the base flange, and arranged in a regular pattern relative to each other.

Embodiments may relate to a Tuohy-Borst adapter, comprising a main valve body comprising an inner luminal wall extending from a distal opening to a proximal opening, and defining a lumen; the foregoing dynamic radial seal mountingly received by a seat of the main valve body; a sidearm in fluid communication with the lumen and equipped with a three-way stopcock configured to switch fluid communication between the lumen and one of two alternate fluid paths, wherein a confluence joins the sidearm and lumen in fluid communication; and a pressure transducer protruding through an opening into a lumen of the sidearm, the pressure transducer being isolatable from fluid communication with the lumen by switching the three-way stopcock.

Other benefits and advantages will become apparent to those skilled in the art to which it pertains upon reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, wherein like reference numerals indicate like structure, and wherein:

FIG. 1A is a cross-sectional view of a valve according to an embodiment of the invention;

FIG. 1B is a cross-sectional view of the valve of FIG. 1A receiving a catheter;

FIG. 2 is an exploded view of the valve of FIGS. 1A and 1B;

FIG. 3A is a top view of a seal according to an embodiment of the invention;

FIG. 3B is a side view of the seal of FIG. 3A;

FIG. 3C is a bottom view of the seal of FIG. 3A;

FIG. 3D is a second side view of the seal of FIG. 3A;

FIG. 3E is an elevation view of the seal of FIG. 3A;

FIG. 3F is a cross-sectional view of a double conical seal combining two conical gaskets;

FIG. 4A is a top view of an embodiment showing the purge valve and the alternative flow paths in relation to the three-way stopcock;

FIG. 4B is a side cross-sectional view showing the length of the saline line and its confluence with the lumen of the main valve body;

FIG. 5 is a cross-sectional view of a cap-type purge valve taken through the purge valve perpendicular to the lumen of the main valve body;

FIG. 6 is a cross-section taken through the purge valve parallel to the lumen of the main valve body;

FIG. 7 is a perspective view of the embodiment;

FIG. 8 is a cross-sectional view of a plunger-type purge valve taken through the purge valve perpendicular to the lumen of the main valve body;

FIG. 9 is a cross-sectional view of a second Tuohy-Borst embodiment containing a second radial seal embodiment;

FIG. 10 is a view of the second radial seal embodiment of FIG. 9;

FIG. 11 is a view of the second radial seal embodiment in an unrolled configuration;

FIG. 12A is a semi-transparent view of an embodiment having occlusive elements comprised of perpendicular slits;

FIG. 12B is an exploded view of the embodiment of FIG. 12A;

FIG. 12C is a plan view of a pliable sheet according to one embodiment;

FIG. 12D is a side view of the pliable sheet of FIG. 12C;

FIG. 12E is a side view of a seal according to one embodiment receiving a catheter;

FIG. 13A is a semitransparent view of an embodiment having occlusive elements comprised of perpendicular slits where the positions of the occlusive elements and the cylindrical seal are reversed;

FIG. 13B is an exploded view of the embodiment of FIG. 13A;

FIG. 14A is a drawing of a single-seam conical gasket;

FIG. 14B is a drawing of a annular frustoconical diaphragm;

FIG. 14C is a drawing of an annular frustoconical anti-prolapse member;

FIG. 15A is a side cross-sectional view of a cooperating stack of the members shown in FIGS. 14A-14C;

FIG. 15B is a cutaway view of a cooperating stack of the members shown in FIGS. 14A-14C;

FIG. 16A illustrates the stack of FIGS. 15A-15B receiving a catheter;

FIG. 16B is a cross-sectional view of the stack of FIGS. 15A-15B receiving a catheter;

FIG. 17A is a partially transparent view of a hemostasis valve comprising the stack of FIGS. 15A-15B;

FIG. 17B is an exploded view of a hemostasis valve comprising the stack of FIGS. 15A-15B;

FIG. 18A is a stack similar to that of FIGS. 15A-15B but further includes second single-seam conical gasket offset by 90° from the first single-seam conical gasket;

FIG. 18B is a cross-sectional view of the four-member stack of FIG. 18A;

FIG. 19 is a cross-sectional view of a three-member stack having two conical seals but no diaphragm;

FIG. 20A is a sectional view drawing of a hemostasis adapter according to an embodiment illustrating precompression of a valve element;

FIG. 20B is a side view of an embodiment showing valve elements in precompression sufficient to cause a proximal seam to open; and

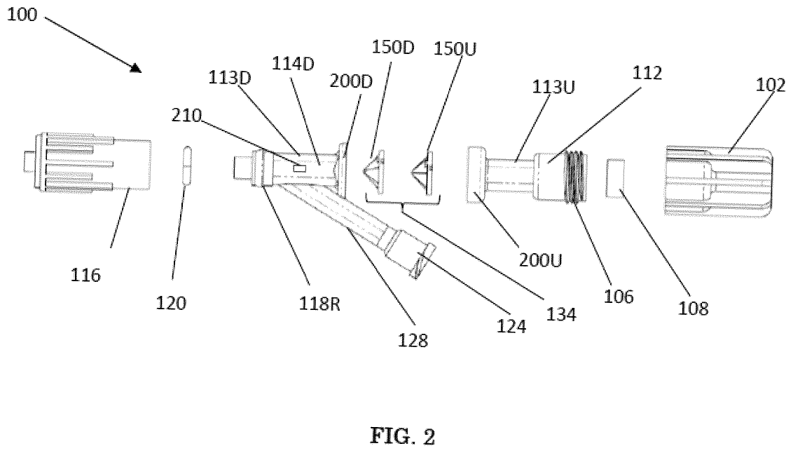

FIG. 2C is a sectional view of a hemostasis adapter with valve elements under precompression and backpressure.

### IV. DETAILED DESCRIPTION OF THE INVENTION

As used herein the terms “embodiment”, “embodiments”, “some embodiments”, “other embodiments” and so on are not exclusive of one another. Except where there is an explicit statement to the contrary, all descriptions of the features and elements of the various embodiments disclosed herein may be combined in all operable combinations thereof.

Language used herein to describe process steps may include words such as “then” which suggest an order of operations; however, one skilled in the art will appreciate that the use of such terms is often a matter of convenience and does not necessarily limit the process being described to a particular order of steps.

Conjunctions and combinations of conjunctions (e.g. “and/or”) are used herein when reciting elements and characteristics of embodiments; however, unless specifically stated to the contrary or required by context, “and”, “or” and “and/or” are interchangeable and do not necessarily require every element of a list or only one element of a list to the exclusion of others.

The terms distal and proximal are used herein to indicate the relative position or orientation of parts of an embodiment in an assembled state, and/or while in use. Their meaning will be clear in context to the ordinarily skilled artisan, but in general they refer to the direction of travel of a catheter as it is inserted into an embodiment.

The term blood pressure and physiological blood pressure is meant to indicate typical pressure regimes encountered in human subjects.

The term “seal” is used herein to mean a sealing surface or collection of sealing surfaces adapted to occlude fluid flow. It is also used herein to denote a discrete member, or a cooperating assembly of discrete members, each having one or more sealing surfaces. Seal is also used in the sense of a discrete member, or collection of members, engaging another element to occlude fluid flow. The person having ordinary skill in the art will readily understand the meaning of seal based on the context provided herein.

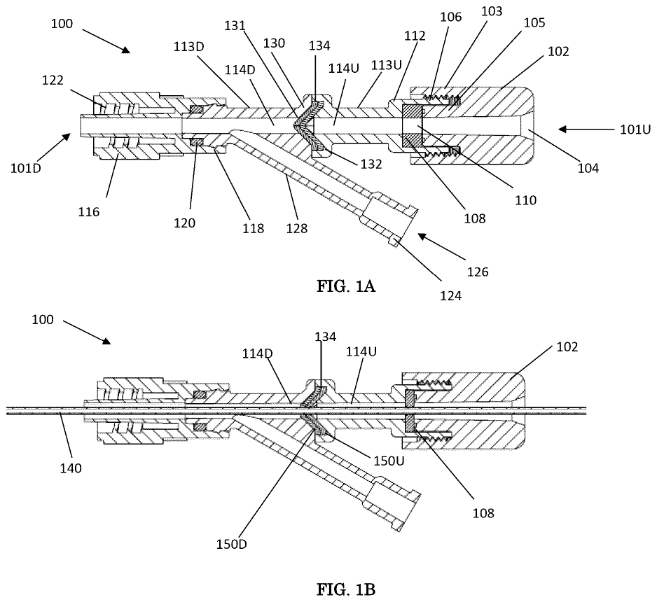

Referring now to the drawings wherein the showings are for purposes of illustrating embodiments of the invention only and not for purposes of limiting the same, FIG. 1A is a cross-sectional view of an embodiment 100 of the invention comprising a fully assembled Tuohy-Borst adapter with an improved dynamic radial seal. The distal end 101U is shown to the right, and the proximal end 101D is on the left. The distal end 101U includes a chamfered, or beveled, catheter access port 104 formed in a compression nut 102. The nut 102 has female threads 103 proximal of the access port 104. The nut also includes a plunger 105 that functions to axially compress a cylindrical gasket 108 as the nut 102 is tightened onto a male thread 106 of the valve body 113U. The cylindrical seal is compressed between the plunger 105 and a seat 112 formed in the valve body 113U. A central through-hole 110 of the cylindrical seal 108 is aligned coaxially with the lumen 114U, 114D which is defined by an inner luminal wall of the valve body 113U, 113D. Thus, a catheter may enter at the distal end, through the access port 104, pass through the through-hole 110, enter the lumen 114U, 114D and exit the valve body at the proximal end 101D of the Tuohy-Borst. The central through-hole 110 is sized to slideably receive the catheter in an uncompressed state. In this context, the term slideably receive means that the catheter is free to travel through the central through hole 110 regardless of whether the catheter actually makes sliding contact with the sides of the through-hole 110.

The valve body is divided into two halves, namely a distal half 113U and a proximal half 113D. The reason for dividing the valve body in this way is to provide structure for easily installing a dynamic radial seal, which in this embodiment is a double conical seal 134, into a mounting groove 132 formed by the two halves. While the present embodiment is divided into two halves, the skilled artisan will readily understand that any of a wide variety of known structures for retaining a seal would also be appropriate as a matter of design choice. Such variations are well within the scope of the present invention as described and claimed herein. FIG. 1A shows a double conical seal 134 held in a mounted relation by the mounting groove 132.

In the embodiment of FIG. 1A, the groove 132 holding the double conical seal has a complex frustoconical-shaped wall 130 formed in the distal and proximal halves of the valve body 113U, 113D. In addition to holding the double conical seal 134 in place, this shape also tends to support a portion of the seal 134 while allowing the apex of the seal to protrude through an orifice 131 and into the proximal lumen 114D. This arrangement may be advantageous by, for instance and without limitation, limiting the amount of flexure that the seal experiences during insertion of a catheter and/or providing improved sealing around a catheter by stiffening the seal and thereby increasing sealing force.

With continued reference to FIG. 1A, the proximal valve body 113D terminates in a rotatable collar fitting comprising an annular ridge and groove connection 118 to a standard Luer Lock fitting 116 threaded 122 to fixedly cooperate with cannulas. By fixedly cooperate, it is contemplated that the threads of the Luer Lock fitting may receive a cannula having complementary structure in a fastened and thus fixed relation relative to the Luer Lock fitting. The fitting 116 is sealed with an O-ring 120 to prevent leakage of fluids from the lumen 114D, 114U. Some embodiments, including the one shown in FIG. 1A, may include a sidearm flush 128 with a port 124 co-operable with standard fluid delivery devices such as syringes. The lumen 126 of the sidearm flush is shown in fluid communication with the proximal lumen 114D of the proximal valve body 113D.

In contrast to FIG. 1A, FIG. 1B illustrates the same embodiment 100 receiving a catheter 140. The catheter is shown locked in place by the cylindrical seal 108 which has been compressed by tightening the nut 102. Accordingly, the seal 108 has collapsed around the catheter 140 and thus locks it in place through friction. FIG. 1B also illustrates the distal conical gasket 150U and the proximal conical gasket 150D of the double conical seal 134 opening at their apexes to receive the catheter 140. The gaskets 150U, 150D dynamically radially seal against the catheter 140 as it is inserted into the embodiment 100 and fed into a patient. The gaskets 150U, 150D then statically maintain the seal when the catheter 140 is locked in place, as shown here.

FIG. 2 is an exploded view of the embodiment shown in FIGS. 1A and 1B. The valve body is shown divided into its distal 113U and proximal 113D halves. The distal valve body 113U includes a seat 112 receiving a cylindrical seal 108. A nut 102 is threaded onto the male thread 106 of the distal valve body 103U, which compresses the cylindrical seal 108 with a plunger 105 (see FIG. 1A). Interposed between the two halves of the valve body 113U, 113D are two conical gaskets. One is a distal conical gasket 150U and the other is a proximal conical gasket 150D. The base of the distal gasket 150U fits into a seat 200 at one end of the distal valve body 113U. The two gaskets 150U, 150D stack one within the other, and their angular orientation relative to each other is set by registering structures, as will be described in more detail below.

The conical gaskets 150U, 150D are mounted between a distal flange 200U and a proximal flange 200D. The distal and proximal flanges 200U, 200D include the frustoconical wall 130 and groove 132 which are not visible in this figure, but which can be seen in FIG. 1A. The proximal end of the proximal valve body 113D terminates in a ridge 118R of the ridge and groove connection 118 shown in FIG. 1A. The ridge 118R receives the Luer Lock collar fitting 116 in a rotatable relation sealed with an O-ring 120.

A pressure transducer 210 is shown mounted within the lumen 114D of the proximal valve body 113D. The transducer advantageously has a thin profile which allows it to be in the lumen without occluding or obstructing. Thus, the transducer cooperates with a catheter 140 in that it does not obstruct its path. Accordingly, the transducer is capable of obtaining real time measurements of body fluid pressures while carrying out a procedure without the need for additional fluidics, and without the need to pause the procedure to measure pressure. Suitable pressure transducers are well known in the art and may be selected as a matter of design choice. Optionally, the transducer 210 may include or communicate with electronic components for wirelessly broadcasting telemetry data. The skilled artisan will appreciate that the placement of the transducer 210 is advantageously within the proximal lumen 114D because the distal lumen 114U is isolated by the double conical seal 134.

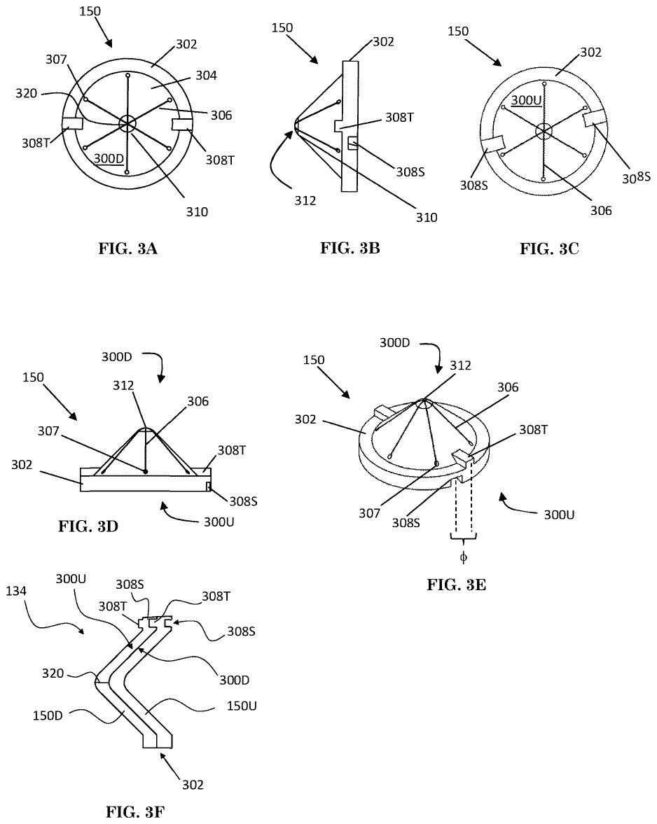

FIGS. 3A through 3E illustrate the same conical gasket 150 in various orientations. The embodiment illustrated in FIGS. 1-2 illustrates a double conical seal 134 which is made of a stacked pair of this conical gasket 150 which, in FIGS. 1-2, are labeled distal 150U and proximal 150D. Their unique reference numbers 150U and 150D are intended only to indicate their position in the assembled device. In this embodiment, the distal and proximal conical gaskets are structurally identical to each other and to the gasket illustrated in FIGS. 3A-3E. The skilled artisan will readily appreciate that being identical is not a requirement, but that certain manufacturing efficiencies are gained by having two of a single part rather than two different parts. Alternatively, the distal gasket and the proximal gasket may be different in that each may limit its registering structures to those configured to engage the other.

With collective reference to FIGS. 3A-3E a conical gasket 150 is shown that has an annular base flange 302. The terms base flange and annular base flange are used throughout the present disclosure to denominate a structure for mounting a seal to a Tuohy-Borst adapter. It will be understood by the person having ordinary skill that the base flange can have other geometries without departing from the scope of the invention. More specifically, the geometry of the base flange must complement that of the adapter so that the two sealingly mate. The geometry of the base flange and adapter can be adjusted to whatever geometry the skilled artisan chooses as a matter of design choice or convenience.

The base flange 302 cooperates with the groove defined in the distal and proximal flanges 200U, 200D of FIG. 2. The distal surface of a conical wall 300U and the proximal surface of the same wall 300D are shown divided into six equal semi-conical flaps 304 through the apex 312. The edges of each semi-conical flap 304 abut the edges of its nearest neighbors to form seams 306. As used in this context, the term seam is intended only to denote area where flap edges abut one another, and it is not intended to imply that the edges are joined. To the contrary, the edges are not joined, and thus the flaps 304 can spread apart in response to an impinging catheter to form an opening at the apex 312 where the catheter may pass through.

The circle 310 is not a structural element of the conical gasket 150. Rather, it is intended to indicate the region where the conical wall 300U, 300D begins to curve to form the blunted apex 312 shown most clearly in FIGS. 3B, 3D, and 3E.

Each seam 306 terminates in a circular through-hole 307 near the base flange 302. This structure is optional, but may be advantageous in preventing tearing of the gasket at the seam terminuses. The gasket 150 has a pair of register tabs 308T located on the proximal surface 180 degrees apart from each other. Similarly, the illustrated embodiment includes a pair of register sockets 308S located on the distal surface 180 degrees apart. Thus, a pair of the gasket 150 may be stacked such that the tabs 308T of one cooperatively fit into the sockets 308S of the other. Register tabs 308T and register sockets 308S are referred to herein according to their genus as register structures, or registering structures. Thus, the angular orientation of the gaskets relative to each other may be fixed.

The skilled artisan will readily appreciate that the number and distribution of register tabs and register sockets may vary. Embodiments may have only one register tab 308T and/or one register socket 308S provided that they are positioned to cooperate with the tabs and sockets of other gaskets 150. Alternatively, embodiments may have a plurality of tabs and sockets, and they may be disposed on either the distal or proximal surface, or even on both surfaces.

With further regard to FIGS. 3A-3E the register tabs 308T and register sockets 308S are shown off-set from each other by an angle φ. The precise magnitude of the off-set is not critical; however, it should be sufficient to cause the seams 306 of two stacked gaskets 150 to be sufficiently off-set from each other to allow the semi-conical flaps 304 to elastically spread under normal operating conditions, where the embodiment is sealably receiving a catheter, without causing bleed-back of body fluids into the distal lumen 114U. In other words, the gap formed by one gasket's spreading flaps is filled by the flap of the adjacent gasket. Suitable magnitudes will depend in part on the number of semi-conical flaps 304, which may be more or fewer than the illustrated number without departing from the scope of the invention. The skilled artisan will appreciate that a greater number of flaps 304 requires more seams 306 which requires smaller angular off-sets. Suitable magnitudes for φ according to the illustrated embodiment include any angle from 1° to 59°. Other ranges within the scope of the invention include 1° to 5°, 5° to 10°, 10° to 15°, 15° to 20°, 20° to 25°, 25° to 30°, 30° to 35°, 35° to 40°, 40° to 45°, 45° to 50°, 50° to 55°, 55° to 59°, or any combination thereof.

The semi-conical flaps 304 discussed above are a species of occlusive elements which cooperate to form a dynamic radial seal that allows a device such as a microcatheter to slide against the flaps while maintaining a fluid barrier preventing the backflow of blood or other body fluids.

With particular regard to FIG. 3F, a cross sectional view of a double conical seal 134 is shown comprising an upstream conical gasket 150U and a downstream conical gasket 150D. The cross section is taken so as to show the registering socket 308S of the downstream conical gasket 150D receiving, i.e. engaging, the registering tab 308T of the upstream conical gasket 150U. As shown, the upstream conical wall 300U of the downstream conical gasket 150D abuts the downstream conical wall 300D of the upstream conical gasket when the registering structures, namely the registering tab and registering socket, of the respective conical gaskets engage each other. The registering socket 308S and tab 308T are shown as part of the annular base flange 302. This view also shows that the openings 320 of the upstream and downstream conical gaskets 150U, 150D are aligned.

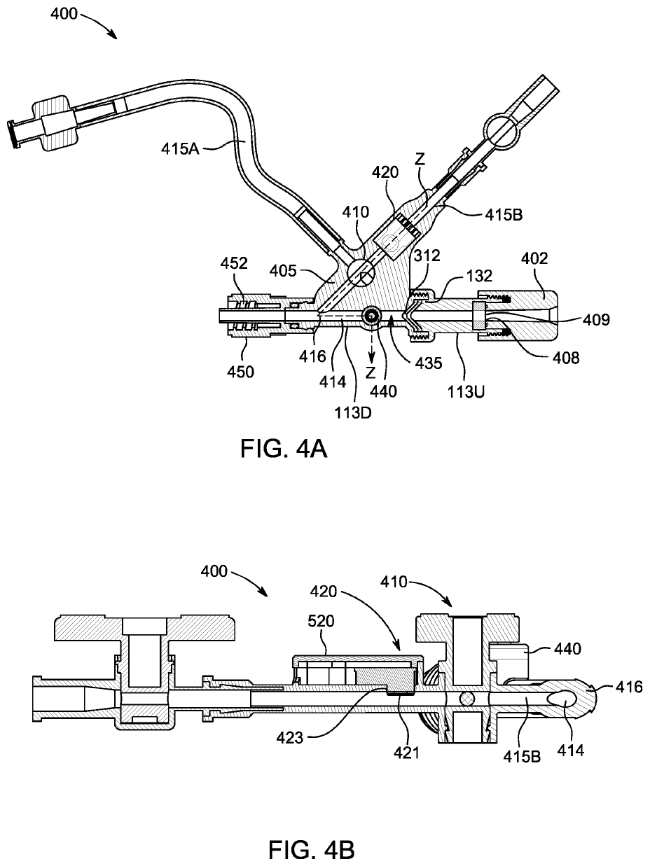

FIG. 4A illustrates an embodiment 400 having a three-way stopcock 410. The stopcock 410 provides fluid communication between the lumen 414 and two alternate fluid paths 415A and 415B. The illustration of the three-way stopcock 410 in FIG. 4A, is intended to show the internal fluid paths provided by the stopcock. The stopcock itself is indicated by circle 410. The external parts of the stopcock, such as the handle, are omitted from the drawing to clearly reveal the internal structure. According to this embodiment the lumen 414 can only communicate with one alternate fluid path 415A, 415B at a time. The stopcock 410 switches communication with the lumen 414 between the alternate paths 415A, 415B. Therefore, a device, such as pressure sensor 420, is isolatable from fluid communication with the lumen 414 by switching the three-way stopcock 410. The alternate fluid paths 415A, 415B may supply any of a wide variety of known fluids including, without limitation, saline or contrast dye.

With continuing reference to FIG. 4A, the valve embodiment 400 includes a cylindrical seal 408 disposed upstream of the conical gaskets and having a central through-hole 409 aligned and in fluid communication with the lumen 414. The seal 408 is compressible and operates similar to seal 108 of FIGS. 1A and 1B. More specifically, the seal 408 can be compressed by tightening a threaded compression fitting such as nut 402. The nut 402 axially compresses the cylindrical seal, which causes the central through-hole 409 to collapse around a device, such as a catheter (see FIG. 1B), passing through the central through-hole 409. The threaded nut 402 applies a continuously adjustable compressive force to the cylindrical seal, allowing the user to control the degree of compression by turning the nut 402. The central through-hole 409 of the cylindrical seal 408 is sized to slidably receive a catheter when the seal 408 is in an uncompressed state, and to lockably receive a catheter in when the seal 408 is in a compressed state. As used herein slidably receive means to allow a catheter or other device to move axially in the through-hole 409 regardless of whether the catheter and cylindrical seal 408 actually make contact. Also as used herein, lockably receive means that the cylindrical seal 408 collapses around the catheter or other device within the through-hole 409, holding the device stationary relative to the seal 408 through friction.

Still referring to FIG. 4A, the embodiment 400 includes a threaded mount 450 fixedly co-operable with a mountable needle, cannula, trocar, sheath, introducer, or similar structure (not shown). The threaded mount 450 may be, without limitation, a standard Luer lock fitting as shown in FIG. 4A. As used here, fixedly co-operable means that the threads 452 of the fitting 450 cooperate with complementary structures of e.g., a needle, to fix the orientation of the needle relative to the threaded mount 450.

A purge 440, as shown in FIG. 4A, may be useful when a catheter (not shown) or similar device inadvertently introduces air bubbles into the lumen. Should the air be carried into the patient's blood, a potentially deadly air embolism may result. Thus, it is advantageous to have a structure like purge 440 for clearing air bubbles. The purge 440 may comprise a variety of known structures that permit the out-flow of liquid for the purpose of expelling or purging air bubbles from the region 435 near the tip, or blunted apex 312, of the double conical seal 134 (see FIG. 1). Thus the purge 440 is positioned near the tip 312. A source of purge fluid may be, for instance, the saline line 415B. In the illustrated design, the saline would flow along net-flow path Z, from the saline sidearm 415B, through the lumen 414, and out the purge 440. A portion of the saline would flow past the purge 440 cause turbulent flow in the region 435, causing air bubbles to be swept away from the tip 312 and out through the purge 440.

FIG. 4B shows the embodiment 400 in a cross-sectional view cutting axially through saline line 415B. The saline line 415B is shown extending horizontally to a confluence 416 with lumen 414. As used herein, the term confluence means a structure where two or more fluid flows meet and form a single flow. A pressure transducer 421 is shown within a housing 422 and protruding through an opening 423 into the lumen of the sidearm saline line 415B. The pressure transducer 421 is isolatable from the lumen 414 by turning the three-way stopcock 410.

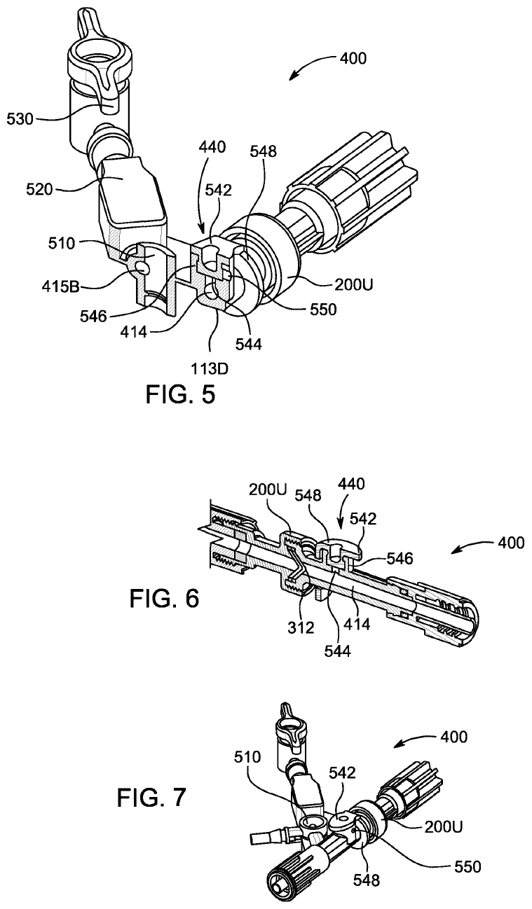

The purge 440 is shown in greater detail in FIG. 5 which illustrates a purge 440 comprising a cap 542 press-fitted into a groove 546 in the valve body. The cap 542 is also referred to herein as a plug. The cap 542 comprises a pliable material such as a silicone rubber or other known medical grade elastomer. The groove 546 holds the cap 440 in sealing contact with a purge orifice 544, defining a sealing surface, which communicates with the lumen 414 and the exterior, i.e. atmospheric pressure. The purge 440 can be opened by pulling the cap 542 from the groove 546, thereby allowing the lumen 414 to communicate with the exterior air pressure through the orifice 544. According to FIG. 5, an embodiment may include a port 550 on the side of the purge 440. The port 550 of embodiment 400 is closed when the cap 542 is fully inserted in in the groove 546. Thus, a user may partially withdraw the cap 542 from the groove 546 such that fluid is expelled through the side port 550 without the need to fully remove the cap 542. Allowing the cap 542 to remain partially inserted in the purge 440 housing permits faster re-sealing.

The cap 542 may be connected to the body of the valve 400 through a retaining ring 548, which prevents loss of the cap 542 and allows for quick replacement of the cap. The purge 440 operates by engaging a pressure source with the lumen 414 while opening the purge 440. For instance, the saline stopcock 530 can be switched on and the cap 542 can be pulled from the groove 546. The saline line 415B is shown exiting the base of the pressure transducer housing 520. The confluence 416 where saline line 415B and the lumen 414 meet is cut away from FIG. 5, but is shown in FIG. 4A.

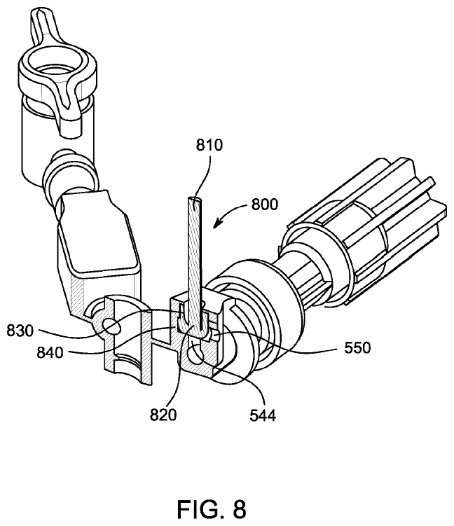

While the embodiment 400 is described in terms of a removable cap, the person having ordinary skill in the art will readily understand that the invention is not limited to such structure, but rather encompasses a wide variety of known structures suitable for closing a purge orifice. For example, and without limitation, pull-tab valves 800 as shown in FIG. 8 are also known in the art. Such valves have a stem 810 which may be pulled to draw a plug, namely plunger 820, upward and away from the orifice 544, allowing fluid to flow out from the lumen through a port 550 in the side of the purge valve 800. Applying an upward force to the stem in turn compresses an annular member surrounding the plug. A spring force generated by an annular member 830 biases the plunger 820 to a normally closed position. The annular member generates a spring force by pushing against an annular interior of the purge valve housing 840.

FIG. 6 shows the embodiment 400 of FIG. 5 is a cross-sectional view through the length of the lumen 414. The purge 440 is shown with the cap 542 in a press fit with the groove 546 being held in sealing contact against the orifice 544. The retaining ring 548 is shown encircling the downstream half of the valve body 113D. A conical seal is shown within seat 200U with the tip, or blunted apex 312, near the purge orifice 544. As used herein, the term near in the context of positioning the purge valve, means that opening the purge tends to communicate with and draw fluid from the volume surrounding the blunted apex, including entrained air or debris such as clots. A purge valve orifice 544 is near the blunted apex 312 provided that opening the purge has the desired effect of expelling entrained air and/or debris. Thus, the purge 440 illustrated in FIG. 6 may be closer or farther away from the blunted apex 312 without departing from the scope of the invention provided that it produces the desired effect, as will be readily ascertainable by the person having ordinary skill in the art without undue experimentation. FIG. 7 illustrates the same embodiment 400 in a perspective view rather than sectional and shows the sidearm and stopcocks that were cutaway in FIG. 6.

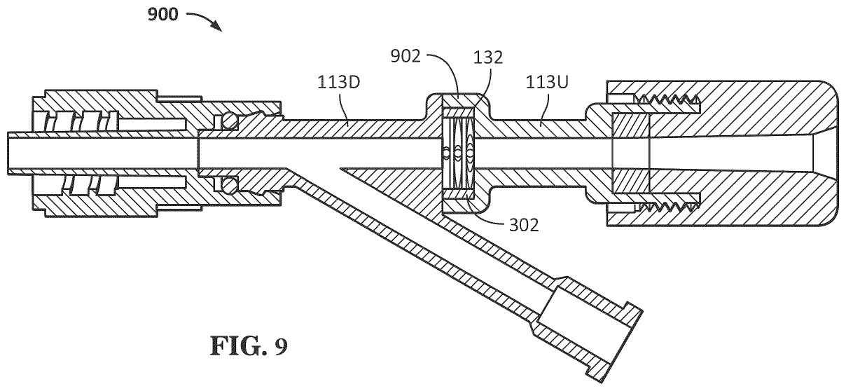

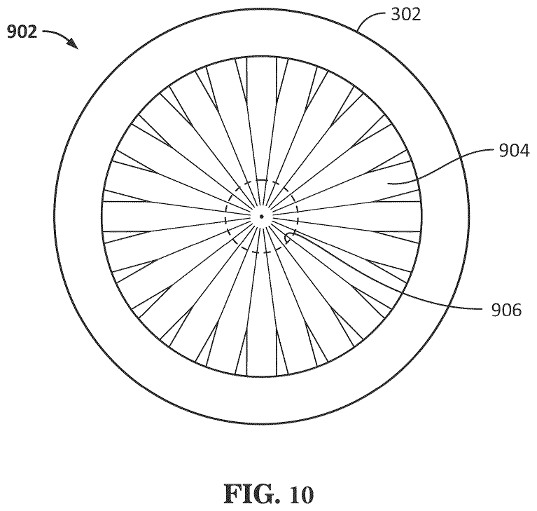

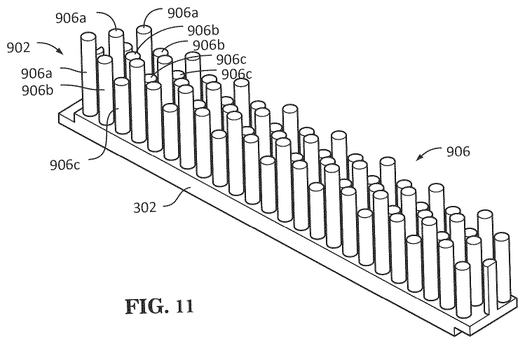

FIG. 9 shows a second Tuohy-Borst adapter embodiment 900. The difference between this and embodiment 100 is radial seal 902. Radial seal 902 operates similarly to that of the double conical seal discussed above; however, the flaps are replaced by cilia, as shown more clearly in FIGS. 10 and 11. FIG. 10 shows that the seal 902 includes an annular base flange 302 adapted to engage a mounting groove 132 formed by the distal half 113U and a proximal half 113D of the Tuohy-Borst embodiment 900. The annular base flange has an annular planar surface oriented radially inward. In other words, as shown in FIGS. 10 and 11, the base flange is the equivalent of a flat surface rolled into an annulus. Other embodiments need not have a planar annular surface. Rather, such surface may be replaced by a secondary curvature perpendicular to the curve of the annulus. Such embodiment may take on a toroidal form, or a complex form having characteristics of a torus. In the illustrated embodiment, the annular planar surface is oriented radially inward in the sense that a line drawn normal to the surface would pass through a center of the seal 902 where the seal is essentially circular. This description of the orientation of the surface is not meant to limit the invention to perfectly circular embodiments or perfectly regular surfaces. The person skilled in the art will readily understand that embodiments are operational within tolerances as the skilled artisan can determine as a matter of design choice.

The seal 902 further comprises a plurality of inward-facing cilia 904 which function similar to the semi-conical flaps 304 of other embodiments herein. Unlike the semi-conical flaps 304, the inward facing cilia 904 are directed more or less radially inward rather than at an oblique angle. Therefore, as shown in the side view of FIG. 9, the seal 902 is a flat annulus rather than conical in shape when viewed side-on. FIG. 10 shows that the cilia converge in a central location 906 within the annulus 302, which occludes the fluid pathway that would otherwise be present inside the annular base flange. Furthermore, the breaking pressure of the occlusion is above physiological blood pressure. As used here, the term breaking pressure means the pressure where a seal is overcome. The person having ordinary skill in the art will understand that breaking pressure is typically represented as a range about an average e.g., 300 mmHg +/−10%. Additionally, the breaking pressure is that which is measured in blood. Breaking pressure varies with the liquid that the seal is challenged to resist due to effects of viscosity, surface tension, polarity and other physical factors.

When a microcatheter is inserted through the seal embodiment 902, the seal impinges radially upon the catheter thus forming a seal. Moreover, the seal is effective even when the microcatheter is moving axially through the Tuohy-Borst, relative to the seal 902. Axial motion is enabled by the small radial sealing force, which produces relatively little friction, leaving the catheter free to move while maintaining a dynamic seal. The sealing force is limited in part by the Youngs Modulus of the material composing the cilia, which is typically a silicone, although the invention is not limited to silicones. The cilia are pliable structures that bend easily under shearing forces like that of an axially impinging catheter. The small elastic constant tending to resist flexure is enough to hold back blood under physiological conditions. Meaning, though the elastic constant is small, a patient's blood pressure is smaller. Therefore, the stiffness of the pliable cilia is enough to resist blood flow under both static and dynamic sealing conditions.

Similar to the semi-conical flaps 304 discussed elsewhere herein, the cilia 904 are another a species of occlusive elements which cooperate to form a dynamic radial seal that allows a device such as a microcatheter to slide against the cilia while maintaining a fluid barrier preventing the backflow of blood or other body fluids.

Turning to FIG. 11, a seal embodiment 902 is shown unrolled. The base flange 302 is severed in this view, allowing the cilia 906 to all face in the same direction. According to FIG. 11, the cilia are different lengths. The function of variable length is to avoid overcrowding the cilia, which would otherwise cause them to bunch up, creating areas of excess material that could result in binding up the catheter, or tearing or coring of the pliable cilia by the catheter. Such excess material can be problematic. Therefore, the cilia lengths are staggard so that each longest cilium is surrounded by shorter cilia. More specifically, as shown in this particular embodiment, starting at the far left, a row of cilia starts with a longest cilium 906a, followed by an adjacent medium cilium 906b, and then a short cilium 906c. This pattern repeats through the entire row of cilia. As used here, the term medium cilium means a length intermediate between the longest and the shortest cilia. It is not intended to limit medium cilia to a mathematical median or average or any other specific length. Further, length is meant to indicate its full length from the base of a cilium at the annular planar surface to its tip.

An adjacent row begins at the far left of FIG. 11 with a short cilium 906c which is out of view, and the pattern repeats as in the first row. Therefore, the rows are offset by one cilium. The third row starts with a medium cilium 906b, which is also out of view, and the pattern repeats, thus incrementing the offset so that the third row is offset by two cilia from the first row. This pattern of staggering lengths is effective in mitigating overcrowding of the cilia and allows the catheter to pass through the seal 902 without coring the seal, and without excessive resistance. With respect to specific lengths, the actual length of the cilia will depend on the bore size of the Tuohy; however, in general, normalizing the lengths to the long cilium 906a, the medium cilium 906b is between 90% and 60% of 906a, and the short cilium is between 90% and 60% of 960b.

The embodiments described herein are examples of a more general invention. A dynamic radial Touhy-Borst seal is provided that comprises two basic components. One is a means for seating, connecting, or mounting a seal within a Tuohy-Borst adapter so that the seal remains fixed in an operative position while acted upon by fluid pressure and microcatheters. The annular base flange is an example of such a means for seating the seal. Another basic component is a plurality of pliable occlusive elements that are arranged in a regular pattern relative to each other. As used here, the phrase “arranged in a regular pattern relative to each other” means that the occlusive elements are ordered in a repeating manner that radiates inwardly within the means for mounting, that they contact each other to form an occlusion, and that they maintain the occlusion as they flex in response to receiving a catheter or similar device. To radiate inwardly means that the occlusive elements are directed toward a central axis, without limiting the invention to a particular angle. Some embodiments, for example, are conical seals where the occlusive elements approach the central axis at an oblique angle. Other embodiments are flat discs where the occlusive elements approach the central axis at a right angle. Occlusive elements that are inwardly directed, or radiate inward, according to the invention are dimensioned and organized to apply a radial force to a device passing through a Tuohy-Borst adapter, such as a microcatheter. Moreover, the radial force is sufficient to seal against a physiological pressure of a liquid, like blood or saline.

The occlusive elements are sufficiently pliable to flex when acted upon by e.g., a microcatheter, allowing it to pass through the occlusion. At the same time, the occlusive elements are sufficiently rigid to resist fluid pressure and thereby block fluid flow through the seal. Furthermore, the seal is dynamic, meaning it forms an effective radial seal against e.g., a microcatheter, even when the microcatheter is moving relative to the seal. The occlusive elements described herein include various arrangements of rubber flaps, overlapping rubber flaps, and cilia. However, the invention is not limited to such structures. Occlusive elements can take other forms as well, provided that they perform the necessary functions of, as noted above, cooperating with a Tuohy-Borst adapter, and dynamically sealing against blood flow at physiological pressures.

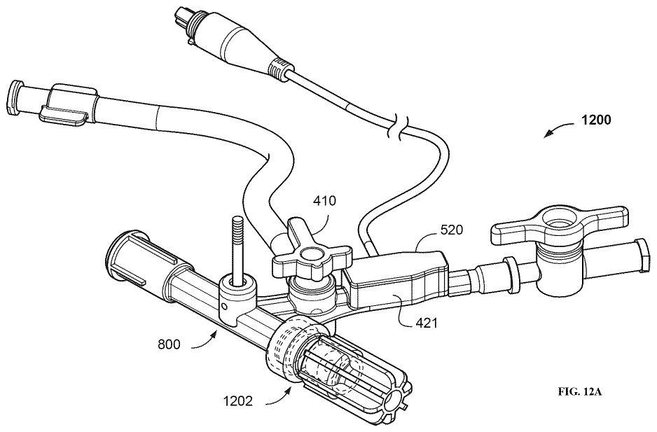

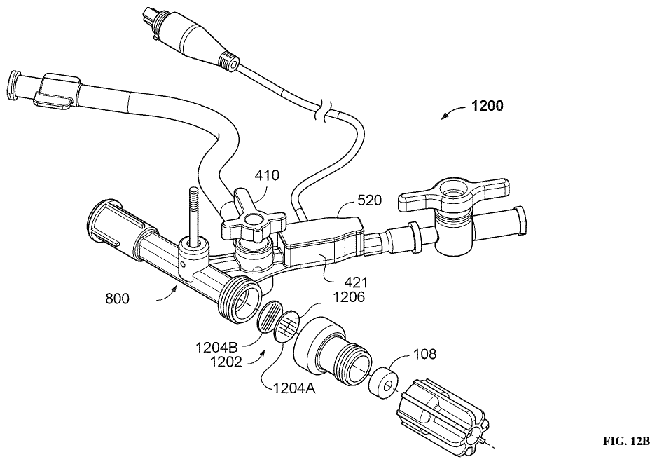

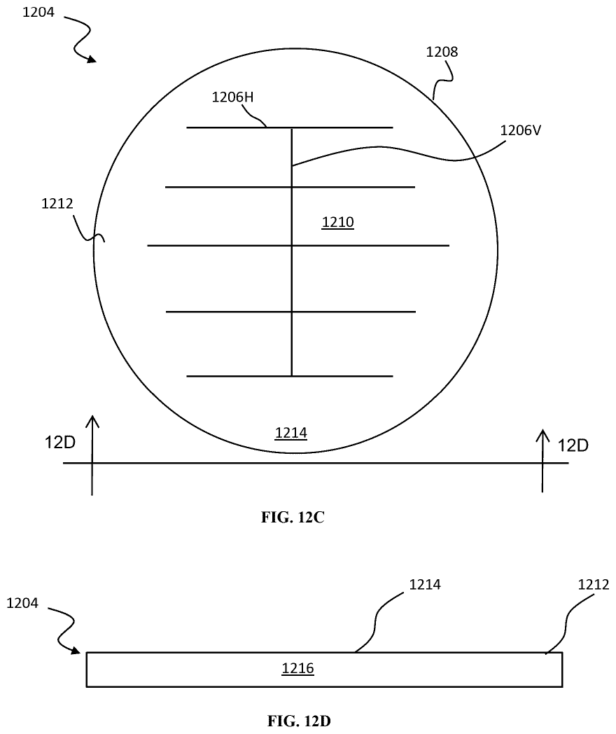

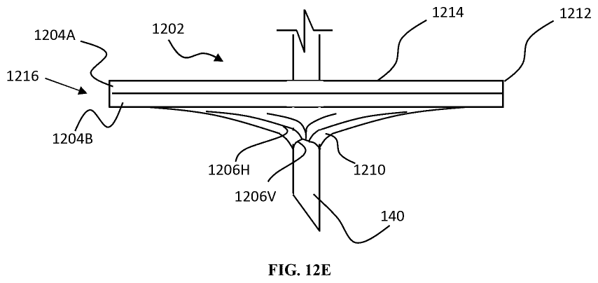

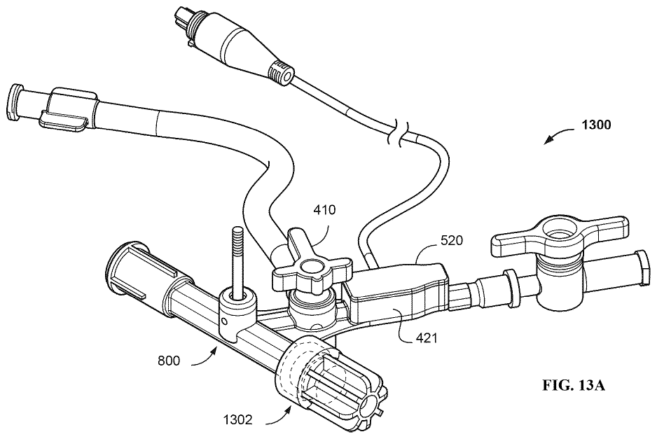

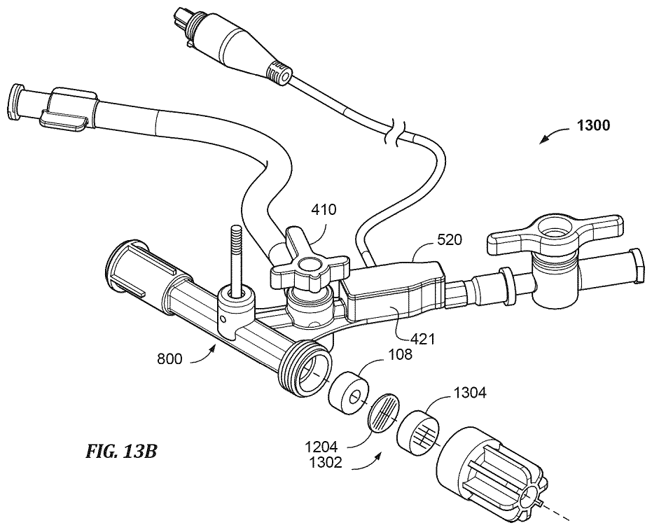

In general, a dynamic radial seal according to the invention has pliable occlusive elements that direct a force radially inward sufficient to hold back liquids at physiological pressures and temperatures. More specifically, such a dynamic radial seal seals interfaces where the occlusive elements contact each other, and where the occlusive elements contact another device, such as a microcatheter or other interventional device introduced with Tuohy-Borst adapters. In some embodiments, the occlusive elements are all contained on a single seal, but in other embodiments, such as those shown and described in connection with FIGS. 12A through 13B the occlusive elements are contained on a complementary pair of seals in sealing contact. As used here, the term complementary pair means that together the two sealing elements complete a single seal, or occlusion. Conversely, alone, each sealing element may be insufficient to form a reliable occlusion. The nature of the complementarity is that as a complementary pair of seals (also referred to as sealing elements) receives a microcatheter, the occlusive elements of each will tend to spread, which would form gaps, or fluid paths, defeating the seal except that the structure of one sealing element fills the gaps formed in the other. Thus, they complement each other. The person having ordinary skill in the art will readily understand the full scope of the terms “occlusive elements” and complementarity upon reading and understanding the teachings set forth herein. Moreover, the person having ordinary skill will readily understand how to combine the various types of occlusive elements and sealing elements taught herein to form seals.

FIGS. 12A through 12D illustrate another embodiment 1200 where the seal 1202 comprises adjacent pliable sheets having slits oriented perpendicular to each other. FIG. 12A is a semitransparent view of an assembled device 1200 showing the placement of the seals 1202 within the embodiment. The exploded view provided in FIG. 12B shows the seal 1202 comprising adjacent pliable sheets 1204A and 1204B oriented at about 90 degrees to each other. Structures 1204A and 1204B are interchangeable and indistinguishable, and are therefore also referred to herein simply as structure 1204. The suffixes A and B are applied to structure 1204 only for convenience to show the relative positioning of two otherwise identical structures.

FIGS. 12C and 12D show structure 1204 in isolation. FIG. 12C is a view looking down upon face 1214, while FIG. 12D is a view looking edgewise at rim 1216 of the base flange 1212. The term base flange as applied to embodiments 1200 and 1300 means the area near the edge of the pliable sheet that is received by a seat in the Tuohy-Borst adapter. Unlike other embodiments herein that have a well-defined base flange structure, visually distinguishable from adjacent parts of the seal without reference to a seat, in embodiments 1200 and 1300 (FIGS. 12A through 13B), the base flange 1212 is only distinguishable from the rest of face 1214 by its contact with a seat in the Tuohy-Borst adapter. Consequently, the definition of the base flange 1212 is dependent on the seat; therefore, changing the dimensions of the seat redefines the base flange 1212 without making a structural change to the base flange 1212 or the pliable sheet 1204.

The pliable sheet structure 1204 is made circular 1208 as a matter of convenience to complement the conventional circular morphology of a Tuohy-Borst adapter's lumen, however, the perimeter shape may vary without departing from the invention. The slits 1206 of the pliable sheet 1204 include a single vertical slit 1206V and a plurality of horizontal slits 1206H. In this embodiment the number of horizontal slits is five. Arranging the slits 1206V, 1206H in this way forms eight finger-like rectangular flaps 1210. The terms vertical and horizontal, as used in this context, are not intended to require a particular orientation. Rather, they are merely meant to describe the relative orientation of the slits as shown in FIG. 12C.

FIG. 12E shows seal 1202 comprising a pair of pliable sheets 1204A, 1204B operably arranged adjacent to one another. Their slits are partially out of view, however, the slits are understood to be oriented at about 90 degrees to each other. The seal 1202 is shown receiving a catheter 140 in a dynamic sealing relation whereby the finger-like flaps 1210 flex to permit the catheter 140 to pass through the slits 1206V, 1206H. It will be understood by the person of ordinary skill that the seal 1202 is operably installed in a Tuohy-Borst adapter, though the surrounding structure is omitted for the sake of visualizing the seal 1202. Furthermore, while only the slits 1204H, 1204V and flaps 1210 of pliable sheet 1204B are visible, it will be understood by the skilled artisan that those of pliable sheet 1204A similarly flex and are in sealing contact with those of 1204B. Since the slits of 1204A are rotated about 90 degrees from those of 1204B, they form a crosswise pattern the slits of 1204B, which complements and reinforces the sealing action of 1204B. In the illustrated embodiment, no registering structures are provided for fixing the relative rotational orientation φ of the pliable sheets 1204A, 1204B; however, other embodiments may include such registering structures. In embodiments such as the one illustrated here, the pliable sheets would be positioned without the aid of registering structures such that they are rotationally offset forming a crosswise pattern. As used here, the term crosswise pattern, as used herein in reference to the relative orientation of pliable sheets 1204 (or 1304 with reference to embodiment 1300), means any angular offset that provides a breaking pressure above physiological blood pressure under both static and dynamic sealing conditions. Such orientations are not limited to 90 degrees or orientations close to 90 degrees. Rather, the degree of offset will depend on material properties such as pliability and hydrophobicity. The person having ordinary skill in the art will be readily able to determine an appropriate offset without undue experimentation.

As shown in FIGS. 12A, 12B, the embodiment 1200 includes a cylindrical seal 108. As discussed herein in relation to other embodiments the cylindrical seal locks a catheter 140 in a fixed axial position within a Tuohy-Borst adapter by compressing the cylindrical seal 108 between a plunger 105 and a seat formed in the valve body (See FIGS. 1A, 1B, and 2). Doing so collapses the seal 108 around the catheter and thus locks the catheter in place through friction forming a static seal.

Also shown in FIGS. 12A, 12B and in FIGS. 13A, 13B are features previously described in relation to other embodiments herein, and now shown in cooperation with the seals 1202 and 1302 of embodiments 1200 and 1300. For example, the pressure transducer housing 520 and transducer 421 are shown installed in a sidearm of embodiments 1200 and 1300, isolatable from the lumen 414 of the main valve body by a three-way stopcock 410. A purge valve 800 is further shown in relation to the seals 1202 and 1302. The person of ordinary skill will readily understand how the features of various embodiments described herein can be recombined to provide embodiments not explicitly shown in the drawings, but which are nonetheless described in enabling detail.

FIGS. 13A and 13B show a related embodiment 1300 where a static radial seal, namely, the cylindrical seal 108 is combined in a single compartment with a dynamic radial seal 1302. The dynamic radial seal 1302 comprises one pliable sheet 1204 like that of embodiment 1200, and one dynamic radial sealing element 1304. Structure 1304 is similar to 1204 except that it is thicker. A related embodiment is identical to embodiment 1300 except that Structure 1304 is replaced with structure 1204.

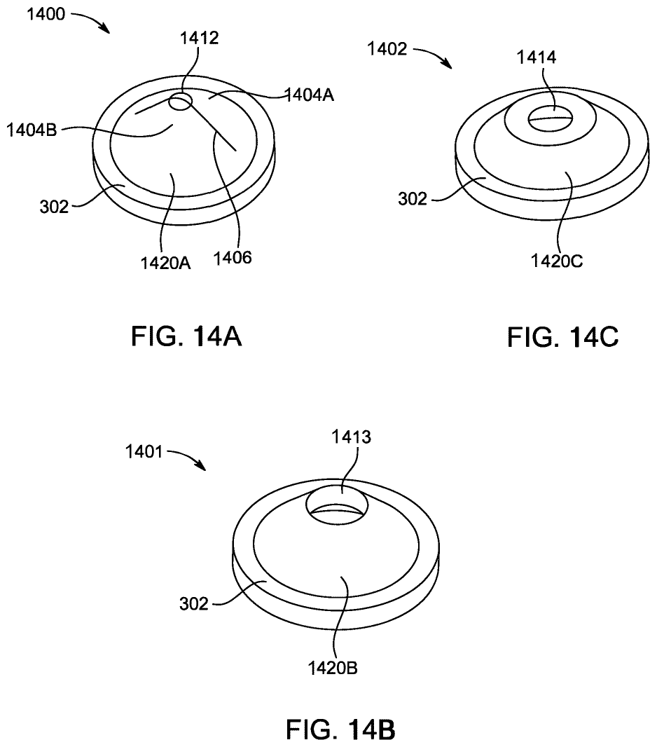

Turning to FIG. 14A, a conical shaped component 1400 is a flexible valve with two flaps 1404A, 1404B separated by a single seam 1406. The seam 1406 is shown passing through a blunted apex 1412. The valve 1400 also includes a base flange 302 which functions as described in relation to FIGS. 3A-3F. While the invention is not limited to material choice, a suitable material is silicone with an approximate hardness of 10-60 Shore A. 20 Shore A is suitable for most embodiments. The valve 1400 can also be fabricated from a suitable thermoplastic elastomer, as the person having ordinary skill in the art will be readily able to select as a matter of design choice without undue experimentation. Registering structures are omitted from this valve 1400 because it is intended for use with the structures shown in FIGS. 4B and 4C which have no seams and therefore do not require an off-set φ.

FIG. 14B is a frustoconical-shaped flexible diaphragm 1401 with a hole 1413 that seals around a cylindrical member inserted through it, such as a catheter (not shown). While the invention is not limited to material choice, a suitable material is silicone with an approximate hardness of 10-60 Shore A. The diaphragm 1401 can also be fabricated from a suitable thermoplastic elastomer, as the person having ordinary skill in the art will be readily able to select as a matter of design choice without undue experimentation.

FIG. 14C is a rigid frustoconical component comprising an anti-prolapse member 1402. The anti-prolapse member 1402 contacts the diaphragm serves to provide structural support to flexible valve 1400 and flexible diaphragm 1401 thereby preventing prolapse of the valve 1400 and/or diaphragm 1401, especially during withdrawal of an instrument, such as a catheter. The anti-prolapse member has a central through-hole 1414 that is slightly larger in diameter than the diameter of the largest device, e.g. a catheter, that may be inserted through it. The slope of the conical wall 1420C of the anti-prolapse member is the same as that of the flexible diaphragm 1420B and the valve 1420A. Thus, the anti-prolapse member 1402, diaphragm 1401, and valve 1400 are structured to be received in a stacked relation, one inside the other, similar to the double conical seal 134 although, unlike the double conical seal 134, no registering structures are required. While the invention is not limited to material choice, a suitable material is stainless steel or a rigid medical-grade plastic, as are commonly used in medical devices. The person having ordinary skill in the art will be readily able to select a suitable material as a matter of design choice without undue experimentation.

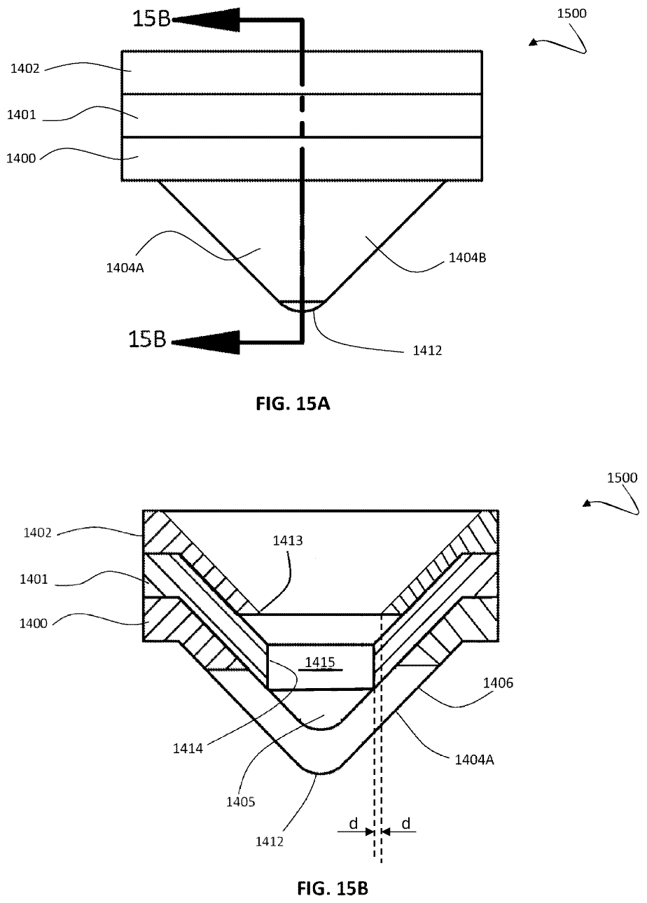

FIG. 15A illustrates a three-member seal stack 1500 according to one embodiment of the invention. The flexible seal 1400 is shown on the bottom of the stack with the blunted apex 1412 pointed downward. The seal 1400 includes two flaps 1404A, 1404B separated by a seam. The seam is not visible in this view because it overlays line 15B-15B indicating the cross-sectional view shown in FIG. 15B. The flexible diaphragm 1401 stacked on top of the seal 1400, and the anti-prolapse member 1402 is stacked on top of the diaphragm 1401.

Turning to FIG. 15B, the anti-prolapse member 1402 is shown at the top of the stack 1500 with its central through-hole 1413 opening downward. The flexible diaphragm 1401 is positioned directly under the anti-prolapse member 1402 and directly above the seal 1400. The central through-hole 1414 of the flexible diaphragm is coaxially positioned relative to the central through-hole 1413 of the anti-prolapse member 1402. Additionally, the hole 1413 of the anti-prolapse member 1402 is slightly wider than the hole 1414 of the diaphragm by a distance “d”. This distance “d” allows for the flexible diaphragm to stretch to sealingly receive a device such as a catheter (not shown) that is slightly larger than the hole 1414 of the diaphragm 1401. Thus, the catheter stretches the hole 1414 around its diameter, creating a seal. The hole 1413 of the anti-prolapse member 1402, being somewhat larger than the hole 1414 of the diaphragm 1401, slidably receives the catheter while maintaining clearance between the side of the hole 1413 and the catheter. As the catheter proceeds downward through the sealing stack 1500 it contacts the inside (upstream) surface 1405 of the valve 1400, separating the flaps 1404A and 1404B at seam 1406 and exiting through the seam 1406 in the area of the blunted apex 1412. As shown, the faces of the three members 1400, 1401, and 1402 directly contact each other. No registering structures are necessary since only the single valve member 1400 has a seam 1406.

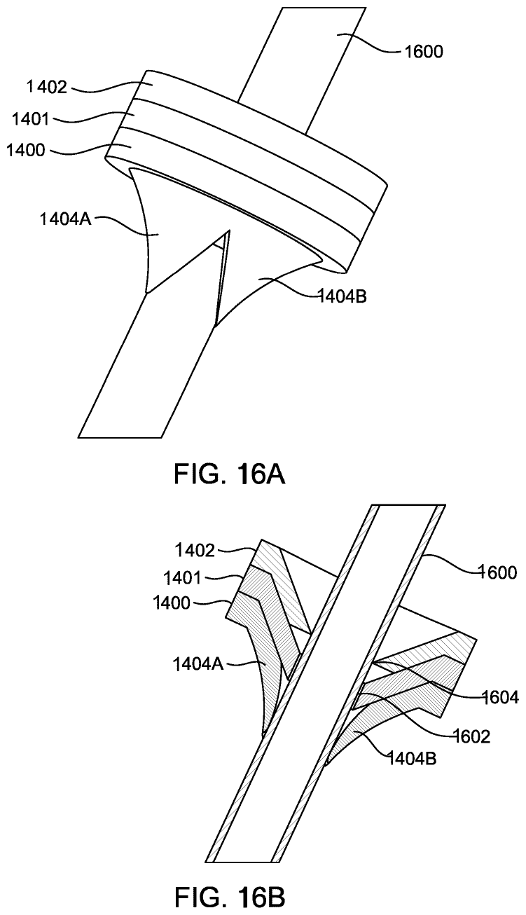

FIG. 16A shows the three-member valve stack 1500 with a catheter 1600 shaft inserted to show how the valve 1400 opens, separating the flaps 1404A, 1404B. FIG. 16B is a cross section of the open valve 1400 on the bottom with the diaphragm 1401 on top sealing 1602 around the catheter shaft 1600 when inserted. Clearance 1604 is also visible between the catheter 1600 and the side of the hole 1413 of the anti-prolapse member 1402.

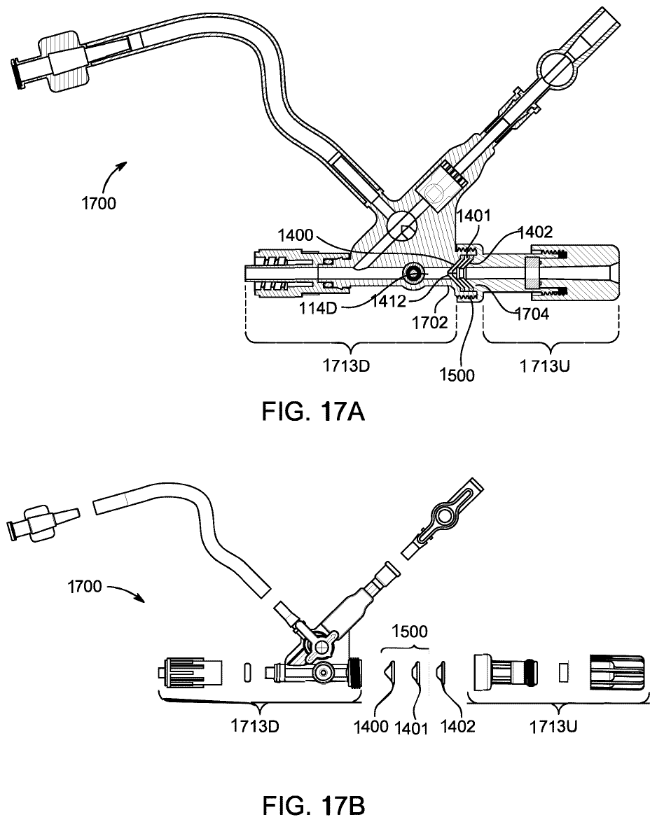

FIGS. 17A and 17B illustrate how the valve stack 1500 cooperates with a hemostasis valve 1700. Similar to other embodiments described herein, the valve body is divided into upstream and downstream halves 1713U, 1713D and the flanges 302 (See FIG. 4A-4C) of the valve stack 1500 are held in compression between a first frustoconical seat 1702 and a second frustoconical seat 1704. The blunted apex 1412 of the valve 1400 protrudes into the downstream lumen 114D.

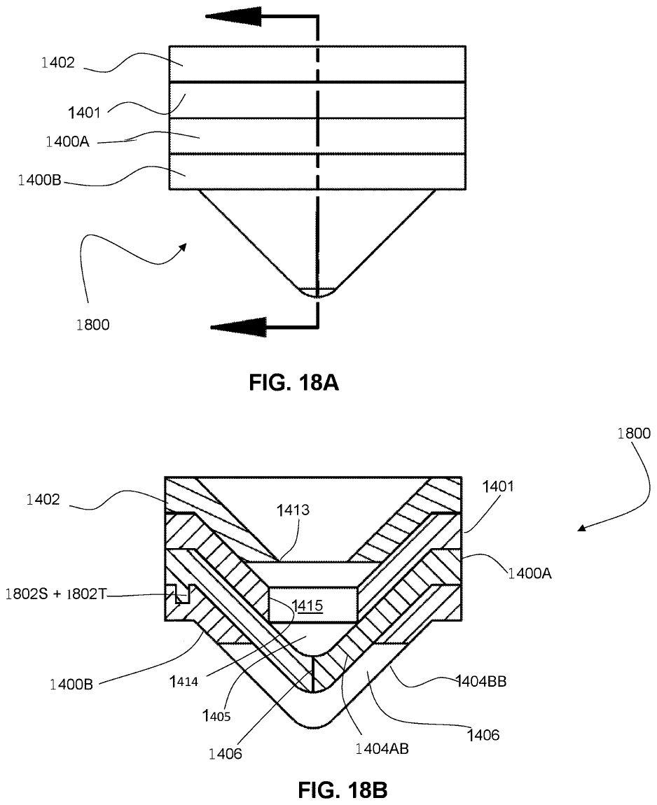

FIGS. 18A and 18B show a four-member valve stack 1800 including a diaphragm 1401, an anti-prolapse component 1402, and a pair of single-seam gasket seals 1400A, 1400B with the seams oriented at 90° to each other. The seams are not visible in FIG. 18A, but are shown in FIG. 18B. The seams 1406 of both seals 1400A, 1400B are shown. The seam 1406 of seal 1400A is represented by a line because the seam 1406 is perpendicular to the page, while seam 1406 of seal 1400B is illustrated parallel to the page. Since the valve stack 1800 is shown in cross-section the visible portion of the seam 1406 of seal 1400B is the portion that is defined by flap 1404BB. A corresponding flap 1404AB of seal 1400A is shown rotated 90 degrees. Stack 1800 is similar to stack 1500 except that it includes two seals 1400A, 1400B with seams 1406 turned 90 degrees relative to each other. The angular offset is set by registering structures described previously herein. Specifically, seal 1400A has a tab 1802T that is received by a corresponding socket 1808S of seal 1802S. Optionally, the seals may be structurally identical by providing tabs and sockets on each seal.

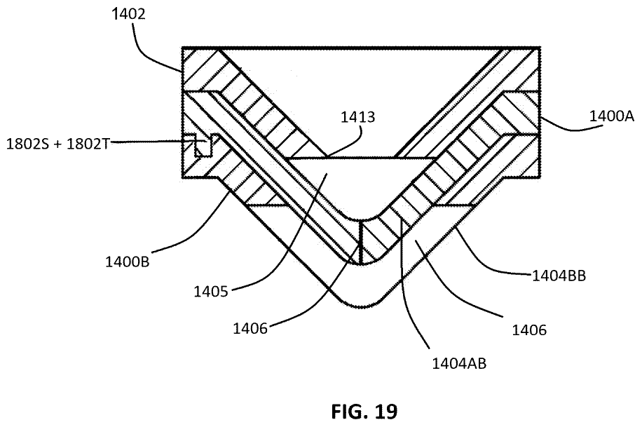

FIG. 19 shows a three-member valve stack embodiment 1900 comprising a pair of single-seam gasket seals 1400A, 1400B with the seams 1406 oriented at 90° to each other, and an anti-prolapse member 1402. This embodiment 1900 is similar to embodiment 1800 except that it omits the diaphragm 1401. The diaphragm 1401 is optional, but may be advantageous for limiting backflow of fluids.

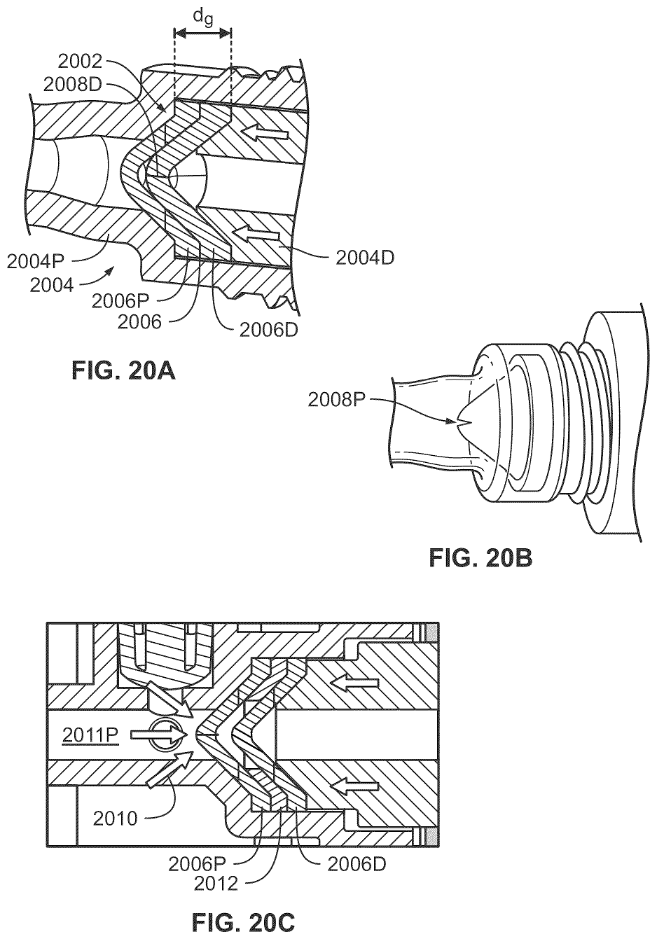

With respect to conical seals as shown in FIGS. 1A, 1B, 2, 3A-3F and as described throughout this specification in relation to various embodiments, sealing is assisted with a combination of preloading (also referred to as precompression) the conical seal and applying an opposing backpressure. Referring now to FIGS. 20A through 20C, a valve stack 2002 is shown installed within a hemostasis valve body 2004. The valve body has two halves, one proximal 2004P to the patient and one distal 2004D from the patient. The valve stack is installed in a groove 2006 formed between the two halves of the valve body. The size of the groove d_{g} is set to provide a preloading or precompression of the valve stack sufficient to cause the seam 2008D in the distal valve 2006D to be squeezed shut as it is compressed into the tapering internal geometry of the proximal valve 2006P. The person having ordinary skill in the art will readily understand how to set a sufficient precompression without undue experimentation including the magnitude of said precompression. In some embodiments the size of the groove (d_{g}) is fixed at the time of manufacture, however, in other embodiments d_{g} may be adjustable, for instance, by threading the halves of the valve body (2004P and 2004D) together.

Under precompression, the proximal valve 2006P serves as a flexible support for the distal valve 2006D. The action of the distal valve pushing against the proximal valve may force the seam 2008P of the proximal valve to flare open under precompression, as shown in FIG. 20B. The valve stack 2002 sealing action is further assisted by applying a back pressure on the proximal side of the stack. As shown in FIG. 20C, a backpressure 2010 is applied at the portion of the proximal valve exposed to the proximal lumen 2011P, which pushes back on the distal valve 2006D, keeping its seam 2008D closed. In some embodiments the patient's blood pressure provides sufficient backpressure. Embodiments may also include a saline flush. In such embodiments, the pressure of the saline flush must overcome blood pressure, and therefore must be slightly above the patient's blood pressure. The person of ordinary skill will readily understand how to integrate such a saline drip line into a hemostasis adapter to provide the desired flushing action.

The functional range of precompression is sensitive to the material from which the proximal and distal valves are made, especially its hardness and thickness, as well as the particular choice of valve stack elements. A stack consisting of a single cone seal is expected to require a different precompression than a stack consisting of two cone seals (see FIG. 20A), and both will require a different precompression than a stack consisting of two cone seals separated by a flexible diaphragm 2012 (see FIG. 20C). The skilled artisan will readily understand that precompression is set empirically on an embodiment-by-embodiment basis. Setting the magnitude of precompression and saline flush pressure is well within the ordinary skill of the art.

It will be apparent to those skilled in the art that the above methods and apparatuses may be changed or modified without departing from the general scope of the invention. The invention is intended to include all such modifications and alterations insofar as they come within the scope of the appended claims or the equivalents thereof. Having thus described the invention, it is now claimed:

I claim:

1. A seal for a Tuohy-Borst adapter, comprising a dynamic radial seal.

2. The seal of claim 1, wherein the dynamic radial seal comprises a plurality of pliable occlusive elements radiating inward, and arranged in a regular pattern relative to each other, wherein each of the pliable occlusive elements contacts a plurality of other occlusive elements.

3. The seal of claim 2 further comprising a base flange, wherein the plurality of pliable occlusive elements radiate inward from the base flange.

4. The seal of claim 2, wherein the plurality of pliable occlusive elements is selected from semi-conical flaps, cilia, or rectangular flaps.

5. The seal of claim 2, wherein the plurality of pliable occlusive elements is adapted to form an occlusion in a liquid flow path with a breaking pressure above physiological blood pressure.

6. The seal of claim 2, wherein the seal comprises a complementary pair of seals in sealing contact with each other.

7. The seal of claim 6, wherein the pair of seals are further adapted to cooperatively form a sealing contact with an interventional element.

8. A Tuohy-Borst adapter, comprising:
a main valve body comprising an inner luminal wall extending from a distal opening to a proximal opening, and defining a lumen;
a dynamic radial seal mountingly received by a seat of the main valve body, the dynamic radial seal having:
a base flange; and
a plurality of pliable occlusive elements radiating inward from the base flange, and arranged in a regular pattern relative to each other, wherein each of the pliable occlusive elements contacts a plurality of other occlusive elements;
a sidearm in fluid communication with the lumen and equipped with a three-way stopcock configured to switch fluid communication between the lumen and one of two alternate fluid paths, wherein a confluence joins the sidearm and lumen in fluid communication; and
a pressure transducer protruding through an opening into a lumen of the sidearm, the pressure transducer being isolatable from fluid communication with the lumen by switching the three-way stopcock.

9. The Tuohy-Borst Adapter of claim 8 further comprising a cylindrical seal disposed proximal of the dynamic radial seal, wherein a central through-hole of the cylindrical seal is aligned and in fluid communication with the lumen.

10. The Tuohy-Borst adapter of claim 9 further comprising a threaded compression fitting in axially compressive communication with the cylindrical seal such that compression of the cylindrical seal is continuously adjustable through turning the threaded compression fitting.

11. The Tuohy-Borst adapter of claim 10, wherein the cylindrical seal is adjacent to and in contact with the dynamic radial seal.

12. The Tuohy-Borst adapter of claim 10, wherein the cylindrical seal is housed in a first compartment and the dynamic radial seal is housed in a second compartment.

13. The Tuohy-Borst adapter of claim 8, wherein one of the two alternate fluid paths comprises a contrast dye inlet or a saline inlet.

14. The Tuohy-Borst adapter of claim 13, wherein the pressure transducer is in fluid communication with the saline inlet and isolatable from fluid communication with the lumen by switching the three-way stopcock.

15. The Tuohy-Borst adapter of claim 8 further comprising a purge valve disposed near the proximal end of the dynamic radial seal, and disposed distal of the confluence.

16. The Tuohy-Borst adapter of claim 15, wherein the purge valve comprises a sealing surface defined by an orifice in fluid communication with the lumen and with an exterior of the main valve body, a plug cooperating with a plug seat to form a normally closed seal.

17. The Tuohy-Borst adapter of claim 8, wherein the plurality of pliable occlusive elements is selected from semi-conical flaps, cilia, or rectangular flaps.

18. The Tuohy-Borst adapter of claim 17, wherein the plurality of pliable occlusive elements is adapted to form an occlusion in a liquid flow path of the base flange with a breaking pressure above physiological blood pressure.

19. The Tuohy-Borst adapter of claim 18, wherein the Touhy-Borst seal comprises a complementary pair of seals in sealing contact with each other.

20. The Tuohy-Borst adapter of claim 18, wherein the pair of seals are further adapted to cooperatively form a sealing contact with an interventional element.

21. The Tuohy-Borst adapter of claim 8 further comprising:
a flexible diaphragm in contact with the pliable occlusive elements, wherein the diaphragm has a central through-hole in coaxial alignment with a fluid path of the pliable occlusive elements; and
an anti-prolapse member in contact with the flexible diaphragm, and stiffening the flexible diaphragm and the pliable occlusive elements to resist prolapse during withdrawal of an interventional instrument.

22. The Tuohy-Borst adapter of claim 8 further comprising an anti-prolapse member in contact with the pliable occlusive elements, and stiffening the pliable occlusive elements to resist prolapse during withdrawal of an interventional instrument.
